(19) United States
(12) Patent Application Publication
(10) Pub. No.: US 2018/0218112 A1
(43) Pub. Date: Aug. 2, 2018

(54) MOLECULAR BASED DECISION SUPPORT SYSTEM FOR CANCER TREATMENT

(71) Applicant: Ittai AMIR, Raanana (IL)

(72) Inventor: Ittai AMIR, Raanana (IL)

(21) Appl. No.: 15/314,950

(22) PCT Filed: Jun. 4, 2015
(86) PCT No.: PCT/IB2015/054234

§ 371 Date: Nov. 30, 2016

Related U.S. Application Data
(60) Provisional application No. 62/007,938, filed on Jun. 5, 2014.

(51) Int. Cl. G06F 19/18; G06F 19/24; G06Q 50/22; G16H 10/40; G16H 10/60

(52) U.S. Cl. CPC G06F 19/18; G06F 19/24; G01N 33/574; G16H 10/40; G16H 10/60; G06Q 50/22

(57) ABSTRACT

A method of determining the statistical distribution of a treatment's benefit to a patient having a positive molecular marker to the treatment, comprising: using general population statistics to determine the positive effect of the treatment on a patient; using general population statistics to determine the conditional probability of a patient with positive marker to react to the treatment; and using the determined positive effect and conditional probability to determine a benefit factor of the treatment to the patient.

### TECHNOLOGY FIELD

The present invention relates to a method of generating treatment prioritized recommendations for cancer patients, based on molecular data and statistical Evidence-Based Medicine.

### CROSS-REFERENCE TO RELATED PATENT APPLICATIONS

This patent application claims priority from and is related to U.S. Provisional Patent Application Ser. No. 62/007,938, filed 5 Jun. 2014, this U.S. Provisional Patent Application incorporated by reference in its entirety herein.

### BACKGROUND

Molecular Medicine strives to understand normal body functioning and disease pathogenesis at the molecular level, which may allow researchers and physician-scientists to use that knowledge in the design of specific molecular tools for disease diagnosis, treatment, prognosis, and prevention.

A considerable amount of molecular data relating to a cancerous tissue is available to physicians today, such as:
HIC panel/Expression profiling
Full chemo-sensitivity assay
“Hot-spots” RT-PCR panel
“Deep”/NG sequencing
“Liquid-biopsy” study (CTCs/n/t-DNA)
Currently, oncologists have three main sources of data to help them recommend a specific treatment to a cancer patient:
The patient's medical records, including cancer type, stage of disease, previous treatments, etc.
Molecular data derived from any of the above assays, which may provide a table, such as depicted in FIG. 1, comprising, for each marker tested:
   The associated treatment
   The molecular assay type, strength and extent
   An indicator showing whether the patient is positive (has a potential to react to the associated treatment) or negative.
Publications of clinical researches showing statistical distributions of the various treatments efficiency among sampled populations. The efficiency is measured in terms of overall survival (OS), remission time (static disease included) (TTP) and response rate (RR), such as depicted IN FIG. 2.

The main drawback of fitting a treatment to a patient using only these available data sources lies in the fact that there is no known way of comparing/prioritizing a number of positively indicated treatments.

The clinical researches are done on molecularly heterogeneous populations, which are sampled according to various parameters which do not include molecular homogeneity, but rather include other parameters such as age, gender, cancer type, tumor stage etc., which are less relevant when it comes to cancerous tumors. Even if molecular homogeneity is included in the sampling parameters the tumor heterogeneity is by far more robust for a clinical trial to contain.

There is need for a method that will predict the probability of a patient to benefit from a treatment and the benefit itself, given that the patient has a positive marker for this treatment.

### SUMMARY

According to an aspect of the present invention there is provided a method of determining the statistical distribution of a treatment's benefit to a patient having a positive molecular marker to said treatment, comprising: using general population statistics to determine the positive effect of said treatment on a patient; using general population statistics to determine the conditional probability of a patient with positive marker to react to said treatment; and using said determined positive effect and conditional probability to determine a benefit factor of said treatment to the patient.

Determining the positive effect may comprise estimating the survival function of patients reacting to the treatment using general population survival function.

According to another aspect of the present invention there is provided a molecular based decision support method for cancer treatment, comprising: performing the method of claim 1 for a plurality of treatments for which a patient has positive molecular markers; and ranking said treatments according to said determined benefit factors.

Ranking may comprise calculating the significance of the difference between efficiencies of the various treatments and determining a resolution cutoff rule for differentiating between various treatments.

According to yet another aspect of the present invention there is provided a system for determining the statistical distribution of a treatment's benefit to a patient having a positive molecular marker to said treatment, comprising: a system server running molecular guided analysis; at least one patients history repository connected with said system server; at least one medical publications repository connected with said system server; and at least one source of molecular laboratory test results connected with said system server, said system configured to supply personally prioritized treatment recommendations to a patient.

### BRIEF DESCRIPTION OF THE DRAWINGS

For better understanding of the invention and to show how the same may be carried into effect, reference will now be made, purely by way of example, to the accompanying drawings.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the accompanying drawings:

FIG. 1 is a table showing molecular data derived from various assays;

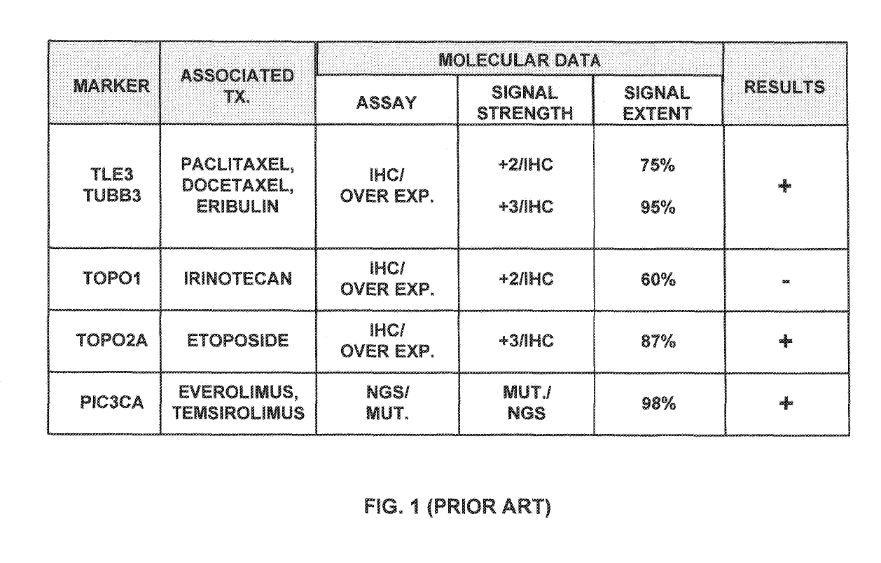

FIG. 2 is a table showing efficiency measurements of various treatments;

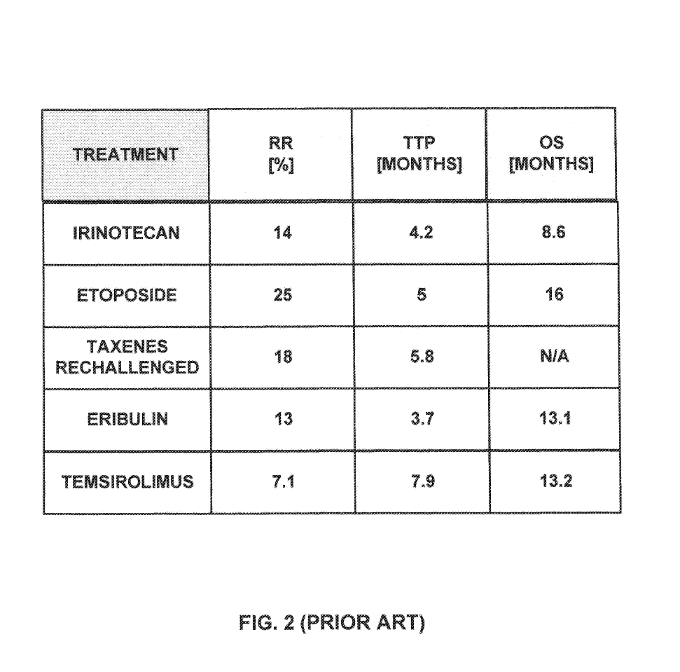

FIG. 3 is a schematic drawing showing the various components participating in the system of the present invention;

FIG. 4 is a general flowchart showing the main processes performed by the molecular guided analysis system;

FIG. 5 shows division of the Kaplan-Meier curve calculated for a given population;

FIG. 6 is a graphic representation of the overall survival;

FIG. 7 shows estimation of RTD from Kaplan-Meier curve; and

FIG. 8 represents the step function for determining the number of different treatments.

### DETAILED DESCRIPTION OF THE INVENTION

In the following detailed description, numerous specific details are set forth regarding the method and the environment in which the method may operate, etc., in order to provide a thorough understanding of the present invention. It will be apparent, however, to one skilled in the art that the present invention may be practiced without such specific details. In other instances, well-known components, structures and techniques have not been shown in detail to avoid unnecessarily obscuring the subject matter of the present invention. Moreover, various examples are provided to explain the operation of the present invention. It should be understood that these examples are exemplary. It is contemplated that there are other methods and systems that are within the scope of the present invention. Also, the same reference numerals are used in the drawings and in the description to refer to the same elements to simplify the description.

The present invention attempts to overcome the shortcomings of existing treatment recommendation methods by deducing, from existing PAB (Population Average Based) statistics, the statistical distribution of a treatment's benefit to a patient having a positive molecular marker to this treatment.

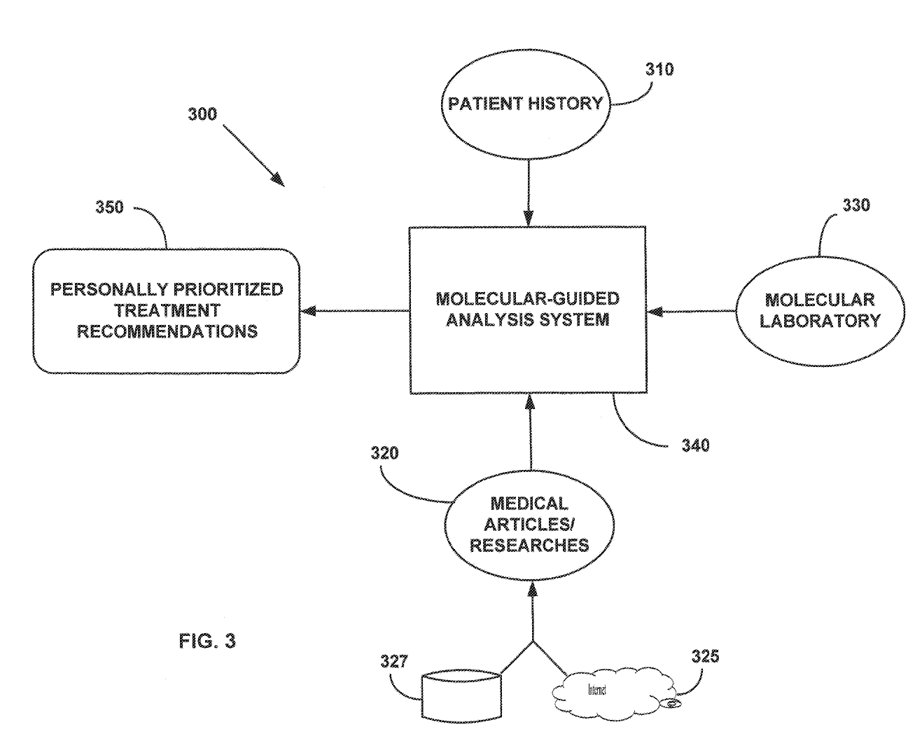

FIG. 3 is a schematic drawing showing the various components participating in the system 300 of the present invention, comprising at least one patients history repository 310, at least one repository of medical publications 320 downloaded from the internet 325 or retrieved from local databases 327 and at least one source of molecular laboratory test results 330, all contributing to the molecular guided analysis system 340 for supplying personally prioritized treatment recommendations 350 to a patient.

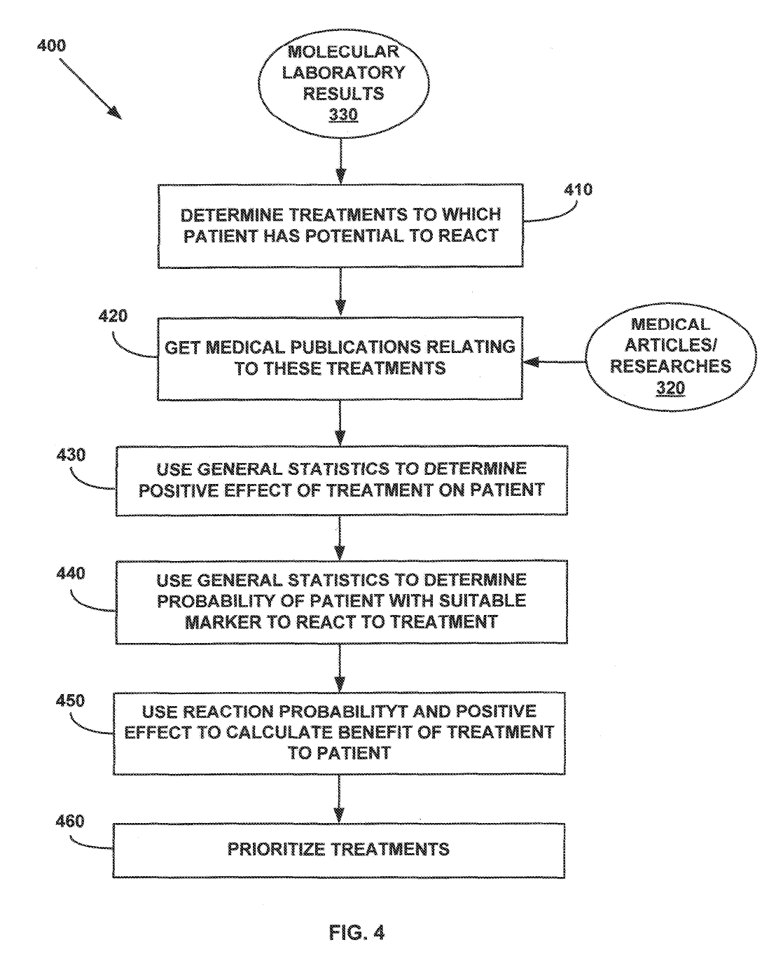

FIG. 4 is a general flowchart 400 showing the main processes performed by the molecular guided analysis system 340.

In step 410 the system determines to which treatments the patient has a potential to react (positive marker) according to the molecular assays results.

In step 420 medical publications relating to these treatments are retrieved from local databases 327 or by ad-hoc internet 325 searches. In step 430 general statistics is used to determine the positive effect of each treatment on the patient, as will be explained in detail below. In step 440 general statistics is used to determine probability of a patient with positive marker to react to each one of the treatment, as will be explained in detail below. In step 450 the general benefit of each treatment is calculated using the previously calculated values and in step 460 the various treatments are prioritized according to the calculated general benefit.

Determining the positive effect of a treatment is done using the Kaplan-Meier estimator. The Kaplan-Meier estimator, also known as the product limit estimator, is an estimator for estimating the survival function from lifetime data. In medical research, it is often used to measure the fraction of patients living for a certain amount of time after treatment. A plot of the Kaplan-Meier estimate of the survival function is a series of horizontal steps of declining magnitude which, when a large enough sample is taken, approaches the true survival function for that population. The value of the survival function between successive distinct sampled observations (“clicks”) is assumed to be constant.

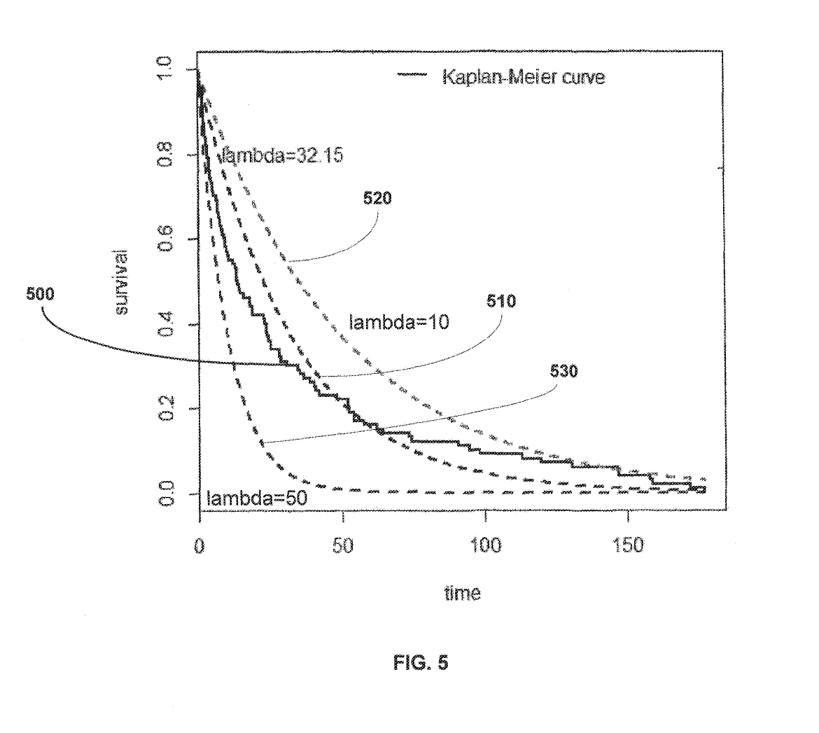

As shown in FIG. 5, the method of the present invention divides the Kaplan Meier curve calculated for a given population, having an exponent fitting curve 510 and a hazard function lambda (e.g. 32.5), into two curves:
A first curve having an exponent fitting curve 520 and a hazard function lambda (e.g. 10), showing survival rate for responders to the treatment (positive marker).
A second curve having an exponent fitting curve 530 and a hazard function lambda (e.g. 50), showing survival rate for non-responders to the treatment (negative marker).

The hazard function lambda is defined as the event rate at time t conditional on survival until time t or later (that is, T≥t).

Survival Function Calculation

In the following text, parameter's notation in cursive capital refers to “true parameters”, and in standard capital to their estimate; e.g. OS is the median Overall Survival of the study's sample and it's the estimate of an unknown “true” median that we will note os.

There is a probability p that the patient will respond after the treatment and a probability (1−p) that the patient will not respond.

Let's call the survival function for patients who respond f0(x) and f1(x) the survival function for patients who don't respond.

Thus, the overall survival function is the mixture:

S(x)=p·f0(x)+(1−p)·f1(x) (1.1)

(f1(x) may be different from survival function of the disease without a treatment because of side effects of the treatment)

We assumed that this relation is true for the medians (It's indeed true for the means but means are less usual in survival analysis, and simulations show that the bias for the mixture medians is not so substantial):

OS=p·RS+(1−p)·NRS (1.2)

OS (Overall Survival) is the median time from the treatment to death/loss of follow up.

RS (Response Survival) is the median time from response to death/loss of follow up.

NRS (Non Response Survival) is the median time from “non-response” to death/loss of follow up.

In oncology p is called RR (Response Rate) and represents patients whose cancer shrinks (termed a partial response, PR) or disappears after the treatment (termed a complete response, CR).

In studies, OS and RR are frequently reported but not RS and NRS.

However there is another parameter which is frequently reported in oncology studies and which may help reproduce RS and NRS:

The TTP (Time To Progression) or PFS (Progression Free Survival) is the median time from the treatment to the progression of the disease.

If we change the definition of “response” in the parameters RR, RS and NRS so that they also include patients with stable disease after the treatment (SD) (and only these ones who have Progressive Disease (PD) are excluded):

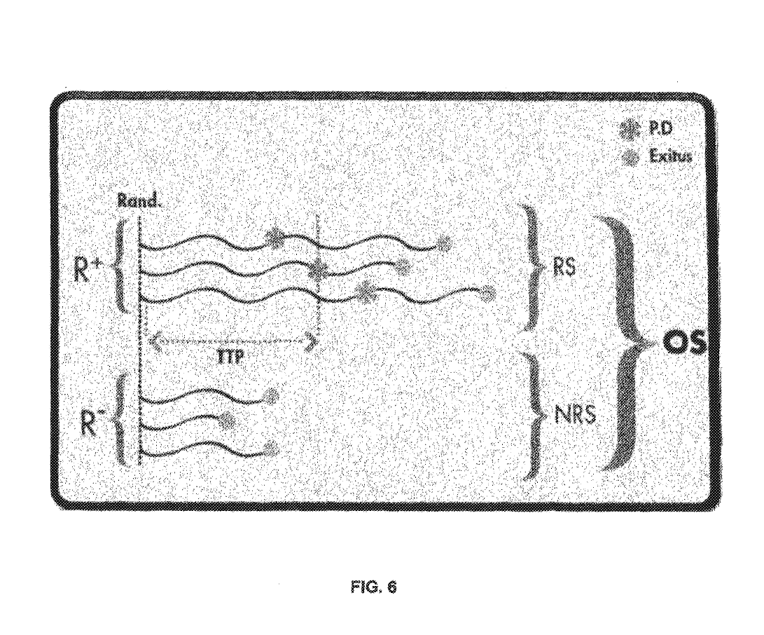

we can assume (See FIG. 6):

RS=TTP+NRS (1.3)

OS=RR×RS+(1−RR)×NRS (1.4)

Hence:

[symbol]

=O [symbol] − [symbol] × [symbol] (1.5)

[symbol]

= [symbol] +O [symbol] − [symbol] × [symbol] =O [symbol] +(1− [symbol] )× [symbol] (1.6)

Where [symbol] , O [symbol] and [symbol] are provided from literature.

RR from the Molecular Results and its Distribution

We now attempt to calculate the conditional distribution of RR, namely the probability of response given an existing marker.

RR is generally given in the study and is a proportion, so n×RR is binomial distributed:

n·RR˜Binom (RR, n) and we get:

$$\begin{matrix}Var ( RR ) = \frac{RR \cdot ( 1 - RR )}{n} \\ So we can compute & ( 2.1 ) \\ sd ( RR ) = \sqrt{\frac{RR \cdot ( 1 - RR )}{n}} & ( 2.2 )\end{matrix}$$

So asymptotically, we get

$$RR ∼ N ( RR , \frac{RR \cdot ( 1 - RR )}{n} ) \qquad ( 2.3 )$$

When we don't know about molecular markers significant results on effectiveness of a treatment (Molecular Prediction of Response=0) we'll use (2.6) for estimating RR.

If we know that a treatment cannot work (Molecular Prediction of Response=−1), we'll admit in a deterministic way that

$$RR = 0 + sd ( RR ) = \sqrt{\frac{RR \cdot ( 1 - RR )}{n}} \qquad ( 2.4 )$$

If we know that a present marker promotes the treatment we can distinguish three cases:

First, let's define the events “the marker exists” Λ+, “the marker doesn't exist” Λ− and the events “the treatment works (including stable disease)” Ω+, “the treatment doesn't work” Ω−.

We make the biologically defendable assumption

Λ−⇒Ω− (or Ω+Λ+) (2.5)

Namely, if the marker doesn't exist the treatment doesn't work (or if the treatment works the marker exists).
If the article provides the conditional probability of the treatment working given that the marker exists P(Ω+|Λ+) we'll use it and its sd is calculable in the same way as normal RR (See equation 2.2)
If we have relevant knowledge on the probability of marker existing P(Λ+) we'll use a Bayesian estimation

P(Ω+|Λ+)=RR/P(Λ+) (2.6)
If we have neither, we'll use in a deterministic way

P(Ω+|Λ+)=RR+α·sd(RR) (2.7)

with α=3 for chemotherapy treatments and 2 for other treatments.

The table below summarizes the estimation of RR for the various cases:

| Summary for estimating | | Molecular Prediction of Response (MPR) | | |
|---|---|---|---|---|
| the parameter RR: | | 1 | 0 | −1 |
| Knowledge | The article | P(Ω+\|Λ+) | RR, | P(Ω+\|Λ−) |
| from | gives | sd | sd(RR) | sd |
| articles | P(Ω+\|Λ+) | calculable | | calculable |
| | other articles | RR/P(Λ+) | | sd(RR) |
| | give P(Λ+) | Without sd | | |
| | no | RR + α · | | |
| | knowledge | sd(RR) | | |
| | | α∈{2,3} | | |
| | | Without sd | | |

The Distribution of TTP, RS, NRS and ART, ARS, APS

We are interested in comparing TTP and RS and NRS from different studies (each study is a different treatment which may concern the patient), but these parameters only make sense when they are adjusted by their probabilities of benefit to the patient.

Let's define new parameters ART, ARS and APS which signify the probability distribution of the various observed values of TTP, RS and NRS:

ART=RR×TTP (3.1)

The probability of a given TTP value given positive reaction to the treatment.

ARS=RR×RS (3.2)

The probability of a given RS value given positive reaction to the treatment.

APS=(1−RR)×NRS (3.3)

The probability of a given NRS value given negative reaction to the treatment.

We assumed that the estimates of TTP, RS and NRS are normally distributed and we can estimate their standard deviations from a confidences interval that appears in the study.

But in survival analysis, software (excepting SPSS) and studies generally use the method of Brookmeyer and Crowley to compute a Confidence Interval (CI) for median overall survival. And this method doesn't use standard deviation at all and gives an asymmetrical CI.

But, as a median, we have:

$$TTP {∼}_{n \to ∞} N ( 𝒯𝒯𝒫 , \frac{1}{n {4 [ f ( 𝒯𝒯𝒫 ) ]}^{2}} ) \qquad ( 3.4 )$$

where f is the unknown TTP's PDF (probability distribution function) and n is its sample size.

To be conservative, we would choose the biggest one half CI (generally the upper one), to estimate the standard deviation, but the methods used by studies, previously conservative (discrete tests dual to CI)

So we defined (by calling henceforth TTP its estimate)

$$sd ( TTP ) = \frac{upCI ( TTP ) - lowCI ( TTP )}{2 ( {z}_{1 - α / 2} )} \qquad ( 3.5 )$$

Where Z is a standard score.

In the case CI is not mentioned in the paper, we assumed that TTP fits an exponential distribution with median=ln(0.5)/λ so f(TTP)=0.5 and asymptotically:

$$sd ( TTP ) = \frac{1}{\sqrt{n}} \qquad ( 3.6 )$$

For RS and NRS we have their estimates from (1.5) and (1.6) and their standard deviations:

Var(NR [symbol] )=Var(O [symbol] −ART)=Var(O [symbol] )+Var(ART)⇒sd(NR [symbol] )=\sqrt{Var(O)+Var(ART)}

Var[(1−RR)×TTP]=Var[RR×TTP]=Var(ART) (3.7)

Var(R [symbol] )=Var(O [symbol] +(RR)×TTP)=Var(O [symbol] )+Var((1−RR)×TTP)=Var(O [symbol] )+Var(ART)=Var(NR [symbol] )

⇒sd(R [symbol] )=sd(NR [symbol] )=\sqrt{Var(O)+Var(ART)} (3.8)

To find the standard deviation of ART=RR×TTP we, conservatively, assumed that RR and TTP are independent and so:

sd(ART)=\sqrt{TTP²sd(RR)²+RR²sd(TTP)+[sd(RR)sd(TTP)]²} (3.9)

And we get a table with the following format:

| | Treatments | | | | | |
|---|---|---|---|---|---|---|
| Articles | TTP | Sd(TTP) | RR | Sd(RR) | ART | Sd(ART) |

Article 1
Article 2
. . .

We can create similar tables for ARS and APS, but in these cases, we don't use twice the sd of RR so we use it, the second time, in a deterministic way:
e.g.

sd(ARS)=RR²·\sqrt{Var(O)+Var(ART)} (3,10)

Treatment Prioritization
There are k possible treatments for the patient and for each treatment we have beforehand estimated by meta-analysis three AEV (Adjusted Efficiency Values), namely:

ART=MPR×RTD,effOS=MPR×RS+(1−MPR)×NRS,subjOS=ARS or APS
(The description of these AEV and the model is found in appendix 1 below).
Similarly, we have already estimated the standard errors of the different estimators assuming a normal distribution and conservatively assuming independence across them.
The aim of this algorithm is not to provide objective rating of treatments but only to rank them using relative grades: The highest score for the most efficient treatment will always be 10 and the lowest score will always be 1 (Naturally, this doesn't allow comparing two treatments of two different reports).
The “Total Efficiency Score” is a weighted average of 3 AEV grades with the following weights:
ART 50%
effOS 45%
subjOS 5%

In this main text we'll describe how the algorithm rates the 3 AEV. Since the procedure is exactly the same for each, we'll describe it once using the generic notation AEV, while the program executes it three times for ART, ARS and APS.

Trivially, we could maintain the original ranking of the AEV estimators regardless their standard errors, i.e. a-priory to assume that the efficacy of each treatment is significantly different from the efficacy of any other. The confidence level at which the whole ranking is true is bound below by 1/(k!) (probability of random guessing). This bound is very small and we can still improve it (see below), but in our work, bounding accurate probabilities by considering statistical hypothesis testing for the difference among any two treatment efficiencies, would be a very challenging task to perform. So we have chosen to consider only the vector of “Confidence Level of the Ranking” for each pair of successive treatment efficiencies (denoted from now on as “treatments”, bearing in mind that we refer to their efficiencies scores). Let's denote the vector of probabilities of having efficiencies difference (which reflects the probability of measuring the efficiency of treatment “A” to be larger than the efficiency of treatment “B”, given the averaged efficiencies and the standard deviations of both treatments) among any adjacent treatments by “CLR”. Looking at its basic properties, it is clear that each of the vector's k−1 components is bound below by 50%, as a result of random guessing (we will see later, how to compute it and we shall use this primary ranking to initialize the algorithm only). On this “random guessing” limit, the low confidence level allows the ranking to be well defined (however an intolerable confidence of this ranking is involved), reflecting k, well ranked, treatment groups of order 1. The prediction regarding the rank of the treatments efficiency, however well defined, is useless because its lack of any statistical significance.

Looking at the opposite limit, it could be argued that any two treatments are absolutely different (statistically-wise), if and only if their CLR is 1, hence 1 is an upper limit of those vector components. Imposing a very high confidence demand on a set of treatments, results in lack of any statistical valid difference between those treatments, or, in other words, having one (unranked) treatment group of order k. Such a predication is also useless to anyone.

Between those extreme limits of CLR, we would want to split the treatments to some different “treatment groups” of one or more treatments in each, respecting the original ranking while also respecting the significance of the standard errors, so that we'll get a vector of Y treatment groups (2≤Y≤k−1) and within each treatment group the difference between the different treatments is not statistically significant (in the sense that will be presented here below).

The purpose of the algorithm is to find a “not-too-small” number of treatment groups, which allows a “not too small” average CLR. If high resolution is required for the ranking (i.e. a large number of treatments groups), the average CLR will be decreased. Similarly, the number of treatment groups will be decreased if high reliability is required for the ranking (i.e. a large average CLR). We use the term “resolution” referring to the number of treatment groups and we use the term “reliability” referring to the average CLR. The value of the resolution parameter is defined over the set {2, 3, . . . , k} since a single group of treatments is not a pertinent result. We consider the “resolution's range” by the interval [2,k], and similarly we define below a relevant “reliability's range”. Given a set of treatment efficiency values, the aim of our algorithm is to find a CLR range such that both the resolution's range and the reliability's range are maximal.

Initializing the algorithm (or step (0)):

The algorithm, as a whole, is illustrated by an example in Appendix II.

We have an ordered vector of k AEV estimators

(AEV₍ₜ₎⁽⁰⁾,AEV₍₂₎⁽⁰⁾, . . . ,AEV₍ₖ₎⁽⁰⁾)

And its standard-errors vector:

(\hat{σ}(AEV₍ₜ₎⁽⁰⁾),\hat{σ}(AEV₍₂₎⁽⁰⁾), . . . ,\hat{σ}(AEV₍ₖ₎⁽⁰⁾))

(We have also the corresponding sample sizes vector (n₁⁽⁰⁾, n₂⁽⁰⁾, . . . , nₖ⁽⁰⁾)

At each step, we also calculate AEG (Adjusted Efficiency Grades) with its standard-error:

AEG is a vector of grades such that to the higher AEV component the equivalent AEG component is 10 and to the lower AEV component the equivalent AEG component is 1.

$$\begin{matrix}{AEG}_{( i )} = \frac{9 \cdot ( {AEV}_{( i )} - {AEV}_{( 1 )} )}{{AEV}_{( k )} - {AEV}_{( 1 )}} + 1 & ( 1.1 ) \\ \hat{σ} ( {AEG}_{( i )} ) = \frac{9 \cdot \hat{σ} ( {AEV}_{( i )} )}{{AEV}_{( k )} - {AEV}_{( 1 )}} & ( 1.2 )\end{matrix}$$

where (1.1) is the projection on the interval [1,10] and (1.2) comes from σ(aX+b)=aσ(X)

For each pair of successive (AEV₍ₜ₎⁽⁰⁾, AEV₍ᵢ₊₁₎⁽⁰⁾) we define δ₍ᵢ₎⁽⁰⁾=AEV₍ᵢ₊₁₎⁽⁰⁾−AEV₍ᵢ₎⁽⁰⁾ and we compute the probability to find a difference between the AEV estimators larger than or equal to δ₍ᵢ₎⁽⁰⁾ when the true difference is null. This probability is naturally the lower bound of the probability to find a difference larger than or equal to δ₍ᵢ₎⁽⁰⁾ as a function of the true difference when this true difference is negative or null (i.e. when the ranking is false). In classical hypothesis testing, for the null hypothesis “the ranking is false” this probability is called the confidence level of the test. Those probabilities form a vector of order k−1 which we denote as “Confidence Level of the Ranking”: CLR⁽⁰⁾

Assuming normal distribution of the estimators, with standard deviation parameters known to be exactly the standard errors estimated, we have:

$${CLR}_{i}^{( 0 )} ≅ Φ ( \frac{{AEV}_{( i + 1 )}^{( 0 )} - {AEV}_{( i )}^{( 0 )}}{\sqrt{{\hat{σ} ( {AEV}_{( i )}^{( 0 )} )}^{2} + {\hat{σ} ( {AEV}_{( i + 1 )}^{( 0 )} )}^{2}}} ) \geq 0.5$$

Where ≤Φ denotes the CDF (Cumulative Distribution Function) of the standard normal distribution:

$$Φ ( z ) = \frac{1}{\sqrt{2 π}} {\int }_{- ∞}^{z} {e}^{- \frac{1}{2} {t}^{2}} dt$$

(We don't use the t distribution because of the wide variety of standard errors which comes from the wide variety of sample sizes among the different treatments. This z-test for comparison of means is spread when there are heteroscedasticity and large sample size)

Then we retain three values:
the smallest component of CLR⁽⁰⁾ called “cutoff CLR⁽⁰⁾” and denoted CLR₍₁₎⁽⁰⁾);
the index of this component l⁽⁰⁾: CLR₍₁₎⁽⁰⁾=CLRₗ_{⁽⁰⁾}⁽⁰⁾);
the mean of the CLR⁽⁰⁾'s components, called “averageCLR⁽⁰⁾” and denoted ξ⁽⁰⁾);

CLR₍₁₎⁽⁰⁾ yields a threshold for the reliability of the whole ranking:

ξ⁽⁰⁾ Is the confidence level of “true ranking” for a pair of successive AEV estimators randomly selected.

The output of the initial step is that:

“For a CLR's threshold in the interval [0.5, CLR₍₁₎⁽⁰⁾], the (maximal) number of treatment groups is k and the average CLR is ξ⁽⁰⁾).”

The General Step (j) of the Algorithm

At each step j, 1≤j≤k−2, we represent two successive “old” treatments (or treatment groups) which bear some different efficiency values in the j−1 step, in one “new” treatment efficiency vales in the j step. This representation is described below (we refer to it by noting that we “pull” treatments' efficiencies and represent it as a different efficiency value):
We are pooling (AEV₍ₗ_{^{(j−1)}}₎^{(j-1)}, AEV₍ₗ_{^{(j−1)}}₊₁₎^{(j-1)}) as if both treatments were a single treatment with an “average Adjusted Efficiency Value”: AEV₍ₗ_{^{(j−1)}}₎^{(j-1)}

From AEV₍ₗ_{^{(j−1)}}₎^{(j-1)}, AEV₍ₗ_{^{(j−1)}}₊₁₎^{(j-1)}, \hat{σ}(AEV₍ₗ_{^{(j−1)}}₎^{(j-1)}), \hat{σ}(AEV₍ₗ_{^{(j−1)}}₊₁₎^{(j-1)}) and using the sample sizes nₗ_{^{(j−1)}}^{(j-1)}, nₗ_{^{(j−1)}}₊₁^{(j-1)} we can represent AEV₍ₗ_{^{(j−1)}}₎^{(j)} and \hat{σ}(AEV₍ₗ_{^{(j−1)}}₎^{(j)}) as follows:

$${AEV}_{( {𝓁}^{( j - 1 )} )}^{( j )} = \frac{{n}_{{𝓁}^{( j - 1 )}}^{( j - 1 )} {AEV}_{( {𝓁}^{( j - 1 )} )}^{( j - 1 )} + {n}_{{𝓁}^{( j - 1 )} + 1}^{( j - 1 )} {AEV}_{( {𝓁}^{( j - 1 )} + 1 )}^{( j - 1 )}}{{n}_{{𝓁}^{( j - 1 )}}^{( j - 1 )} + {n}_{{𝓁}^{( j - 1 )} + 1}^{( j - 1 )}}$$

$$\hat{σ} ( {AEV}_{( {𝓁}^{( j - 1 )} )}^{( j )} ) = \sqrt{\frac{\begin{matrix}{n}_{{𝓁}^{( j - 1 )}}^{( j - 1 )} ( {\hat{σ} ( {AEV}_{( {𝓁}^{( j - 1 )} )}^{( j - 1 )} )}^{2} + {AEV}_{( {𝓁}^{( j - 1 )} )}^{{( j - 1 )}^{2}} ) + \\ {n}_{{𝓁}^{( j - 1 )} + 1}^{( j - 1 )} ( {\hat{σ} ( {AEV}_{( {𝓁}^{( j - 1 )} + 1 )}^{( j - 1 )} )}^{2} + {AEV}_{( {𝓁}^{( j - 1 )} + 1 )}^{{( j - 1 )}^{2}} )\end{matrix}}{{n}_{{𝓁}^{( j - 1 )}}^{( 0 )} + {n}_{{𝓁}^{( j - 1 )} + 1}^{( 0 )}} - {AEV}_{( {𝓁}^{( j - 1 )} )}^{{( j )}^{2}}}$$

The first expression is a simple weighted mean, and the second one comes from the famous decomposition of the empirical variance:

$${σ}^{2} ( X ) = \frac{\sum {x}_{i}^{2}}{n} - {\overline{x}}^{2}$$

true for any sample of a random variable X.

That yields for two samples (x₁, . . . , xₙ), (y₁, . . . , yₘ) for which only the descriptive empirical statistics are known: x̅, y̅, σ_{X}, σ_{Y} the possibility to restore the standard deviation of the samples' union by:

$$\frac{\sum {x}_{i}^{2} + \sum {y}_{i}^{2}}{n + m} - {( \frac{n \overline{x} + m \overline{y}}{n + m} )}^{2} = \frac{n ( {σ}_{X}^{2} + {\overline{x}}^{2} ) + m ( {σ}_{Y}^{2} + {\overline{y}}^{2} )}{n + m} - {( \frac{n \overline{x} + m \overline{y}}{n + m} )}^{2}$$

Obviously we obtain the new sample size: nₗ_{^{(j−1)}}^{(j)}, nₗ_{^{(j−1)}}^{(j-1)}+nₗ_{^{(j−1)}}₊₁^{(j-1)}

The results of this pooling are three new vectors AEV^{(j)}, \hat{σ}(AEV^{(j)})n^{(j)} of k−j components. These “new” vectors are of order which is smaller by 1 compared to the “old” vectors.
We compute the current step CLR by the method mentioned above, that is:

$${CLR}_{i}^{( j )} ≅ Φ ( \frac{{AEV}_{( i + 1 )}^{( j )} - {AEV}_{( i )}^{( j )}}{\sqrt{{\hat{σ} ( {AEV}_{( i )}^{( j )} )}^{2} + {\hat{σ} ( {AEV}_{( i + 1 )}^{( j )} )}^{2}}} )$$

We retain the three following values: the cutoff CLR₍₁₎^{(j)}, its index l^{(j)} and the average CLR^{(j)}:ξ^{(j)}

We conclude for the step j:

“For a CLR's threshold in the interval [CLR₍₁₎^{(j-1)}, CLR₍₁₎^{(j)}], the (maximal) number of treatments groups is k−j and the average CLR is ξ^{(j)}.”

Remark about the Pooling:

We decided to consider only the successive pairs of treatments and not all the pairs because of the algorithm's pooling: Indeed, it may happen that for three successive treatments groups A, B and C the confidence levels of the ranking between A and B and between B and C are larger than the confidence level of the ranking between A and C. In such a case, there is no interest to pool A and C without pooling them with B.

Conclusion and Final Output

The algorithm described two steps functions of the cutoff's intervals. One is the average CLR function overall increasing and the second is the number of treatments groups which is decreasing. The intersection of both functions defines the optimal step J of the algorithm. It may be computed as already stated above, like the maximum of the product function. The product function is the steps function “product of the values on the same scale of both functions”.

To project the two functions on the same scale we use the ranges [2,k] for the number of treatments and the range

$$[ {ξ}^{( 0 )} , {max}_{1 \leq j \leq k - 2} {ξ}^{( j )} ]$$

for the average CLR function.

When the optimal step is not the same for the three AEG we calculate the overall optimum by the weighted mean of the 3 J found.

i.e. J=round(0.5×J_{ART}+0.45×J_{effOS}+0.05×J_{subjOS})

We retain also initial AEG and final AEG (the last step which is “necessary”). But to finally give the Total Efficiency Score (TES) we use the AEG^{(J)}:

TES=0.5×AEG_{ART}^{(J)}+0.45×AEG_{effOS}^{(J)}+0.05×AEG_{subjOS}^{(J)} which is a vector for k−J treatments groups.

### APPENDIX I

Usually, in oncological studies we distinguish three reactions to a treatment well defined as their proportions are called:

ORR (Objective Response Rate which includes Partial Response and Complete Response) based on a percentage reduction of the tumor, PD (Progressive Disease) based on a percentage increase of the tumor and SD (Stable Disease) in which there is neither sufficient shrinkage to qualify for response, nor sufficient increase to qualify for progression.

Our analysis is based on a two group mixture and we called the “responders group” the participants whose tumors did not progress. Their proportion equals to ORR+SD and is called CBR (Clinical Benefit Rate).

From a clinical trial or meta-analysis of several clinical trials on samples, which requires adequate similarities to our patient, we can estimate a naïve probability p for our patient to respond (hereafter, the words “respond” and “response” are based on our definition and include stable disease) to the treatment (and a probability (1−p) that the patient will not respond). The estimation is nave in the sense that we don't use any molecular knowledge specific to our patient.

p=CBR

Let's call the survival function for patients who respond f₀(x) and the survival function for patients who don't respond f₁(x).

Thus, the overall survival function is the mixture:

S(x)=p·f₀(x)+(1−p)·f₁(x) (A.1)

(f₁(x) may be different from survival function of the disease without a treatment because of side effects of the treatment).

We assumed that this relation is true for the medians (It's indeed true for the means but means are less usual in survival analysis, and simulations show that the bias for the mixture medians is not so substantial):

OS=p·RS+(1−p)·NRS (A.2)

OS (Overall Survival) is the median time from the treatment to death/loss of follow up.

RS (Response Survival) is the median time from response to death/loss of follow up in the responders group.

NRS (Non Response Survival) is the median time from the treatment to death/loss of follow up in the non-responders group.

In studies, OS and CBR are frequently reported but not RS and NRS.

However there is another parameter which is frequently reported in oncological studies and which may help reproduce RS and NRS (besides it being an important efficiency value by itself):

The PFS (Progression Free Survival) or TTP (Time To Progression) is the median time from the treatment to the progression of the disease.

In the best case the DCB (Duration of Clinical Benefit) is also reported, which is the PFS of the participants defined like CBR.

In the second best case the Duration of Response is reported, which is the PFS of the participants defined like ORR.

We will show below how we can graphically (and approximately) estimate the DCB from a Kaplan-Meier (KM) curve on PFS or Duration of Response.

We hereafter called the DCB: “RTD”

Let's assume:

RS=RTD+NRS (A.3)

(After the progression a responder is like a non-responder after the treatment)

$$\begin{matrix} Hence \text{:} OS = p \cdot RS + ( 1 - p ) \cdot NRS = p \cdot ( RS - NRS ) + NRS & ( A .4 ) \\ OS = p \cdot RTD + NRS ⇒ RS = \frac{OS - ( 1 - p ) \times NRS}{𝒫} = \frac{OS - ( 1 - p ) \times ( OS - p \times RTD )}{ℛℛ} = \frac{OS - OS + p \times RTD + OS \times p - {p}^{2} \times RTD}{p} = RTD + OS - p \times RTD = OS + ( 1 - p ) \times RTD & ( A .5 )\end{matrix}$$

We use this model twice. In a first time we estimate the general RS and NRS of the sample study with p=CBR. In a second time, if we get a probability to respond less nave than p, called here Molecular Prediction of Response or MPR, we can specifically estimate a more accurate OS for our patient (called here effOS):

i.e. After getting OS, RTD and CBR from the study, we successively compute:

RS=OS+(1−CBR)·RTD

NRS=RS−RTD

effOS=MPR·RS+(1−MPR)·NRS (A.6)

Let's describe how we estimate our RTD from Kaplan-Meier curve:

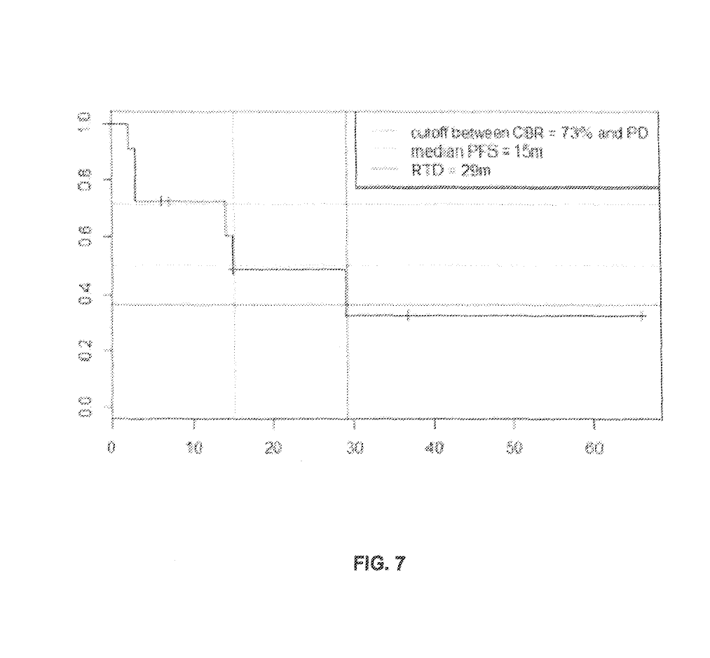

Without taking into account participants who die before progression, the RTD is the median in a KM curve that would ignore the first PD steps: For the graph of FIG. 7, the paper reports PFS=15 m and CBR=73%. So if we remove the top 27% that are PD, the RTD is the median of the remained curve and we consider the survival time of CBR/2, here 0.73/2=0.365 gives 29 m.

Now, the MPR (Molecular Prediction of Response) is based on molecular results of the tumor lab analysis like the following:

At first, we attribute to the patient a “qualitative MPR”: MPR·QI in {−1, 0, 1}

If we don't know about any molecular markers that can predict a clinical response, than: MPR·QI=0

If the molecular analysis predicts no clinical response: MPR·QI=−1, and if the molecular analysis predicts some clinical response (including stable disease) MPR·QI=1.

MPR·QI=0=>we use MPR=CBR

MPR·QI=−1=>we admit in a deterministic way that MPR=0+sd(CBR)

MPR·QI=1=>we distinguish 3 cases.

Before, let's define the events “the marker exists” Λ+, “the marker doesn't exist” Λ− and the events “the treatment works (including stable disease)” Ω+, “the treatment doesn't work” Ω−,

and we assume (it's biologically defendable)

Λ−⇒Ω− (or Ω+⇒Λ+)
In the best case if the article gives P(Ω+|Λ+) we use it.
If we have relevant knowledge on P(Λ+) we use a Bayesian estimation

P(Ω+|Λ+)=CBR/P(Λ+) (2.6)

If we do not have either, we'll use a (heuristic) deterministic estimation

P(Ω+|Λ+)=CBR+α·sd(RR) (2.7)

where α=3 for chemotherapy treatments and 2 for biological treatments.

TABLE 1

| Summary for estimating | | MPR.QI | | |
|---|---|---|---|---|
| the parameter MPR: | | 1 | 0 | −1 |
| Knowledge | The article | P(Ω+\|Λ+) | CBR, | P(Ω+\|Λ−) |
| from | gives | Sd | sd(CBR) | Sd |
| articles | P(Ω+\|Λ+) | calculable | | calculable |
| | other | CBR/P(Λ+) | | sd(CBR) |
| | articles | Sd | | |
| | give P(Λ+) | calculable | | |
| | no | CBR + α · | | |
| | knowledge | sd(CBR) | | |
| | | α∈{2,3} | | |
| | | Without sd | | |

To estimate the standard deviations we use confidence level given in paper for median time and for proportions like CBR we use

$$sd ( CBR ) = \sqrt{\frac{CBR \cdot ( 1 - CBR )}{n}} .$$

The three AEV are finally: the RTD adjusted or ART=RTD·MPR, the effOS and a subjective OS (we choose explicitly in which group the patient is) i.e.

ARS=MPR·RS if MPR·QI=1

APS=MPR·NRS if MPR·QI=−1.

(If MPR·QI=0, the third AEV is not used)

### APPENDIX II

In this appendix, we illustrate the model and the algorithm by a true example.

The treatments alternatives for a patient X and their efficacy estimators reported in clinical trial studies on populations in which the patient could be eligible (according to the exclusion/inclusion criteria) are summarized in the following table:

TABLE 2

| | Treatment | | | | | | | | |
|---|---|---|---|---|---|---|---|---|---|
| | alternative | n | RR % | SD % | CBR % | RTD* months | | OS months | |
| 1 | Raltitrexed | 20 | 0 | | 15 | 4.8 | (2.3-7) | 7.4 | (6-7.8) |
| 2 | Dacarbasine | 68 | 3 | 12 | 15 | 1.9 | | 7.5 | |
| 3 | S-1 | 27 | 7 | 52 | 59 | 2.8 | (0.4-9.7) | 10.5 | (1.7-25.3) |
| 4 | Regorafenib | 505 | 1 | | 41 | 1.9 | (1.6-4.1) | 6.4 | (3.3-12.4 |
| 5 | Cetuximab | 39 | 53.8 | 35.9 | | 6.6 | (4.1-9.1) | 12.5 | |
| 6 | Afatinib | 41 | 11 | 37 | | 3.5 | (1.9-3.9) | 14.2 | (13.2-15.9) |
| 7 | Everolimus | 71 | 0 | 32.4 | 32.4 | 1.8 | (1.7-1.9) | 5.9 | (4.7-7.1) |
| 8 | Tivantinib | 71 | | | 45 | 2.7 | (1.4-8.5) | 7.2 | (3.9-14.6) |

*from TTP or PFS and graphical KM considerations described in Appendix I

The standard errors of the estimators are restored from confidence level and sample sizes (For the CBR proportions the sample size is enough to compute its standard error)

The computation of the RS and NRS is easy from the CBR, TTP and OS estimators by the assumptions RS=OS+(1−CBR)·RTD; NRS=RS RTD explained in Appendix I and we get (in months):

TABLE 3

| Treatment alternative | RS | NRS |
|---|---|---|
| Raltitrexed | 11.48 | 6.68 |
| Dacarbasine | 9.12 | 7.21 |
| S-1 | 11.65 | 8.85 |
| Regorafenib | 7.52 | 5.62 |
| Cetuximab | 13.18 | 6.58 |
| Afatinib | 16.07 | 12.54 |
| Everolimus | 7.12 | 5.32 |
| Tivantinib | 8.69 | 5.99 |

The standard errors are given by the theoretical equations:

Var(NRS)=Var(OS−RTD)=Var(O [symbol] )+Var(RTD)⇒σ(NRS)=\sqrt{σ²(OS)+σ²(RTD)}

Var[(1−CBR)×RTD]=Var[CBR×RTD]=Var(ART)

Var(RS)=Var(OS+(1−CBR)×RTD)=Var(OS)+Var((1−CBR)×RTD)=Var(OS)+Var(ART)=Var(NRS)

⇒σ(RS)=σ(NRS)=\sqrt{σ²(OS)+σ²(ART)}

The molecular predictions of the subject with the appropriated efficacy estimators or prevalence estimator from clinico-molecular and epidemiologic studies give the whole information that we need to obtain the final MPR. See table 1.

TABLE 4

| Treatment | | | | | | |
|---|---|---|---|---|---|---|
| alternative | CBR + α · sd(CBR) | MPR.QI | P(Λ+) | CBR/P(Λ+) | P(Ω+\|Λ+) | MPR |
| Raltitrexed | 30.97 | 1 | 48 | 31.01 | 50 | 50 |
| Dacarbasine | 23.66 | 1 | 46 | 24.42 | 44 | 44 |
| S-1 | 77.93 | 1 | | NA | NA | 77.93 |
| Regorafenib | 41 (CBR) | 0 | | NA | NA | 41 |
| Cetuximab | 4.87 (sd(CBR)) | −1 | | NA | 9 (P(Ω+\|Λ−)) | 9 |
| Afatinib | 71.41 | 1 | | NA | NA | 71.41 |
| Everolimus | 49.06 | 1 | 38 | 53.97 | NA | 53.97 |
| Tivantinib | 62.71 | 1 | | NA | 50 | 50 |

From RTD (table 2), MPR (table 4), RS and NRS (table 3) we get our three AEV by

ART=MPR·RTD; effOS=MPR·RS+(1−MPR)·NRS;

subjOS=MPR·RS if MPR·QI=1 or MPR·NRS if MPR·QI=−1.

Our three AEV are in the following table and constitutes the initialization of the 3 algorithms:

| Treatment alternative | ART | eff.OS | subjOS |
|---|---|---|---|
| Raltitrexed | 0.594 | 6.288 | 3.841 |
| Dacarbasine | 0.779 | 6.4 | 4.011 |
| S-1 | 0.836 | 7.174 | 4.343 |
| Regorafenib | 0.971 | 7.335 | NA |
| Cetuximab | 1.35 | 8.051 | 5.74 |
| Afatinib | 2.182 | 9.08 | 5.988 |
| Everolimus | 2.4 | 11.03 | 9.077 |
| Tivantinib | 2.516 | 15.058 | 11.472 |

The subjOS is not available for Regorafenib because there is no molecular prediction for it.

Let's describe the first steps for one of the three algorithms which have to be done by the ART example:

Step 0

The alternatives (designated here by their references) are ordered by ascending ART, and CLR are computed

(e.g. the first one between treatments 5 and 4 is

$$Φ ( \frac{0.779 - 0.594}{\sqrt{{0.527}^{2} + {0.265}^{2}}} ) )$$

| ref | n | ART | se(ART) | CLR | Average CLR |
|---|---|---|---|---|---|
| 5 | 39 | 0.594 | 0.527 | 62.31 | 58.53986 |
| 4 | 505 | 0.779 | 0.265 | 52.528 | |
| 2 | 68 | 0.836 | 0.859 | 56.199 | |
| 7 | 71 | 0.971 | 0.104 | 65.118 | |
| 8 | 71 | 1.35 | 0.97 | 65.289 | |
| 3 | 27 | 2.182 | 1.881 | 54.088 | |
| 1 | 20 | 2.4 | 0.986 | 54.247 | |
| 6 | 41 | 2.516 | 0.459 | NA | |

Step 1

The smallest CLR is between treatments described at ref #4 and #2 (see above) so we pooled them in a new ART with a new standard error and a new sample size.

The new ART is

$$0.785764 = \frac{505 \cdot 0.779 + 68 \cdot 0.836}{505 + 68}$$

and its new standard error is

$$0.387037 = \frac{505 \cdot ( {0.265}^{2} + {0.779}^{2} ) + 68 \cdot ( {0.859}^{2} + {0.836}^{2} )}{505 + 68} - {( \frac{505 \cdot 0.779 + 68 \cdot 0.836}{505 + 68} )}^{2}$$

| ref | n | ART | se(ART) | CLR | Average CLR |
|---|---|---|---|---|---|
| 5 | 39 | 0.594 | 0.527 | 61.535 | 61.34667 |
| 4 and 2 | 573 | 0.785764 | 0.387037 | 67.803 | |
| 7 | 71 | 0.971 | 0.104 | 65.118 | |
| 8 | 71 | 1.35 | 0.97 | 65.289 | |
| 3 | 27 | 2.182 | 1.881 | 54.088 | |
| 1 | 20 | 2.4 | 0.986 | 54.247 | |
| 6 | 41 | 2.516 | 0.459 | NA | |

Step 2

In the same way we're pooling here treatments 3 and 1

| ref | n | ART | se(ART) | CLR | Average CLR |
|---|---|---|---|---|---|
| 5 | 39 | 0.594 | 0.527 | 61.535 | 63.9058 |
| 4 and 2 | 573 | 0.785764 | 0.387037 | 67.803 | |
| 7 | 71 | 0.971 | 0.104 | 65.118 | |
| 8 | 71 | 1.35 | 0.97 | 69.203 | |
| 3 and 1 | 47 | 2.274766 | 1.567762 | 55.87 | |
| 6 | 41 | 2.516 | 0.459 | NA | |

Step 3

In the same way we're pooling here treatment group 3 and 1 with treatment 6

| ref | n | ART | se(ART) | CLR | Average CLR |
|---|---|---|---|---|---|
| 5 | 39 | 0.594 | 0.527 | 61.535 | 67.362 |
| 4 and 2 | 573 | 0.785764 | 0.387037 | 67.803 | |
| 7 | 71 | 0.971 | 0.104 | 65.118 | |
| 8 | 71 | 1.35 | 0.97 | 74.992 | |
| 3 and 1 and 6 | 88 | 2.387159 | 1.193888 | NA | |

The Last Step (6) Dives:

| ref | n | ART | CLR |
|---|---|---|---|
| 5 and 4 and 2 and 7 and 8 | 754 | 0.846419 | 88.31 |
| 3 and 1 and 6 | 88 | 2.387159 | NA |

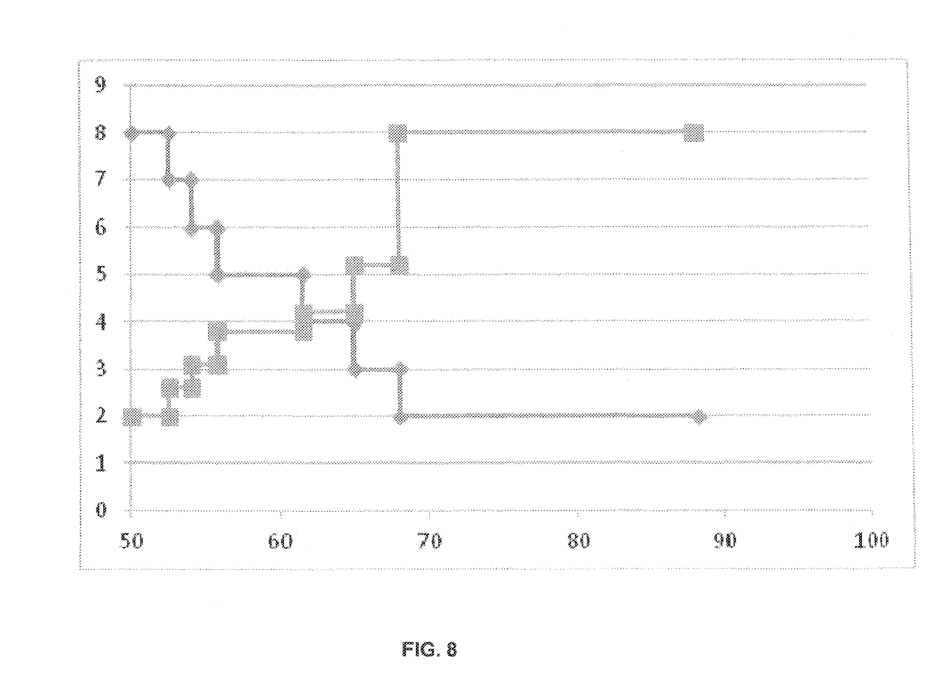

The step functions remaining from the entire procedure are depicted in FIG. 8.

The descending function represents the decreasing number of treatment groups and the ascending function represents the increasing average CLR. The x-axis is constituted by the CLR intervals of the steps and the unit of the y-axis is the interval (2-8) corresponding to the number of treatments and in which the average CLR was projected.

The maximal product is given by step 3 (the intersection in the graph above)

The ART are projected on the grade interval (1,10) and we get:

| | |
|---|---|
| Raltitrexed | 10 |
| Dacarbasine | 2 |
| S-1 | 10 |
| Regorafenib | 2 |
| Cetuximab | 1 |
| Afatinib | 10 |
| Everolimus | 2.9 |
| Tivantinib | 4.8 |

After the 3 algorithms are done we can summarize 3 grades for each alternative and the final grade is the weighted mean of them (The weights are 0.5 for ART, 0.45 for effOS and 0.05 for subjOS when available)

| | grade | grade | grade | |
|---|---|---|---|---|
| Treatment alternative | ART | eff.OS | subjOS | Total.Efficiency.Score |
| Raltitrexed | 10 | 3.8 | 3.2 | 6.7 |
| Dacarbasine | 2 | 2.2 | 1 | 1.6 |
| S-1 | 10 | 5.8 | 7.1 | 7.8 |
| Regorafenib | 2 | 1 | NA | 1 |
| Cetuximab | 1 | 2.2 | 3.2 | 1.2 |
| Afatinib | 10 | 10 | 10 | 10 |
| Everolimus | 2.9 | 1 | 1 | 1.5 |
| Tivantinib | 4.8 | 2.2 | 1 | 3.1 |

1. A method of determining the statistical distribution of a treatment's benefit to a patient, comprising:
using statistics pertaining to the general population to estimate the survival function of patients having received said treatment;
dividing said survival functions into two sub-functions estimating survival properties of responders and non-responders to said treatment, respectively;
receiving molecular data pertaining to said patient, said molecular data comprising at least one molecular marker for said treatment;
assigning said patient to one of a responders group and a non-responders group according to said at least one molecular marker; and
using the appropriate one of said responders survival functions and said non-responders survival functions to calculate said patient's survival probability distributions if given said treatment.

2. (canceled)

3. A molecular based decision support method for cancer treatment, comprising:
performing the method of claim 1 for a plurality of treatments for which a patient has positive molecular markers;
determining a benefit factor of each one of said treatments to said patient, based on said calculated survival probability distributions; and
ranking said treatments according to said determined benefit factors.

4. The method of claim 3, wherein said ranking comprises calculating the significance of the difference between efficiencies of the various treatments and determining a resolution cutoff rule for differentiating between said various treatments.
